(19) United States
(12) Patent Application Publication
(10) Pub. No.: US 2021/0128814 A1
(43) Pub. Date: May 6, 2021

(54) Ternary phased medical procedural process for fracturing a lipid membrane of a viral pathogen that is infecting a patient's pulmonary system and purging the viral pathogen from the patient's pulmonary system (FLiM)

(71) Applicant: Robert George Coghlan, Hialeah, FL (US)

(72) Inventor: Robert George Coghlan, Hialeah, FL (US)

(21) Appl. No.: 17/139,257

(22) Filed: Dec. 31, 2020

(51) Int. Cl. A61M 3/02; A61M 31/00

(52) U.S. Cl. CPC A61M 3/0241; A61M 31/00; A61B 1/2676; A61M 2202/0208; A61M 2210/1039; A61M 2205/15

(57) ABSTRACT

Ternary phased medical procedural process for fracturing and cleansing a viral pathogen from a pulmonary system, (FLiM) of a patient that has tested positive for a pulmonary viral pathogen, and the viral pathogen's constitution consisting of a lipid membrane, for example, the viral pathogen Covid19, by means of a FLiM performed by a Medical Professional (MP), utilizing potential permutations, “MD/S (nPk) x ELT (nPk)” and of at least: a continuous aqueous suspension, herein understood as; a Microcrystalline Castile Colloid (MCC), at least one medical device, a medically equipped operating theater, at least one computer and a network, and comprised of at least the steps of: diagnosing, determining, prepping, performing, diagnosing and following-up on the patient for FLiM medical procedure process.

### CROSS-REFERENCE TO RELATED APPLICATIONS. (N/A)

### STATEMENT REGARDING FEDERALLY SPONSORED RESEARCH OR DEVELOPMENT. (N/A)

### THE NAMES OF THE PARTIES TO A JOINT RESEARCH AGREEMENT. (N/A)

### CORPORATION-BY-REFERENCE OF MATERIAL SUBMITTED ON A COMPACT DISC OR AS A TEXT FILE TO THE OFFICE ELECTRONIC FILING SYSTEM (EFS-WEB). (N/A/)

### STATEMENT REGARDING PRIOR DISCLOSURES BY THE INVENTOR OR A JOINT INVENTOR. (N/A)

### BACKGROUND OF THE INVENTION

In the field of endeavor of the medical arts, a medical process for fracturing a lipid membrane of a viral pathogen that is considered parasitic to a host organism, and whereas the viral pathogen is infecting the pulmonary system of the host organism, in the event of the present invention, the host organism being a primate or Homo sapien, as characterized in the present invention, “patient” and the viral pathogen being comprised of at least Covid19. And whereby the viral pathogen's constitution contains a bilayer composed of a lipid membrane. And whereas a Medical Professional (MP) by utilizing a process of the present invention fractures the lipid membrane of the viral pathogen by means of at least one medical device and a Microcrystalline Castille Colloid, (MCC) continuous aqueous solution and thereafter is rinsed from the pulmonary system of the patient.

### (1) Field of the Invention

The present invention relates to the field of endeavor of a process for providing a medical procedure to a patient that has tested positive for a viral pathogen. And, whereby the viral pathogen is infecting the pulmonary system of the patient, and the viral pathogen is comprised of a lipid membrane, (bilayer). And whereas a ternary phased medical procedural process for fracturing a lipid membrane of a viral pathogen that is infecting a patient's pulmonary system and purging the viral pathogen from the patient's pulmonary system, (FLiM) is practiced upon the patient by a MP.

### (2) Description of Related Art Including Information Disclosed Under 37 CFR 1.97 and 1.98

Prior art with relation to the present invention involves medical techniques, devices, processes, medications and procedures, whereby medical practitioners treat patients that are infected with pulmonary disease, such as Covid19 with forms of air ventilation and life sustaining procedures that provide for the welfare of the patient when the patient is infected with the viral pathogen. Medical practitioners have found some success with these techniques, devices, processes, medications, and procedures. However, no current art taken singularly or in combination provides for a ternary phased medical procedural process for fracturing a lipid membrane of a viral pathogen that is infecting a patient's pulmonary system and purging the viral pathogen from the patient's pulmonary system, (FLiM), that is unified in terms of a MCC and MD/S (nPk) x ELT (nPk) for the purpose of fracturing and purging the inert RNA of the viral pathogen from the patient's pulmonary system. It should be therefore understood, an inventive leap in a process of the present invention provides for such a medical procedural process, and whereby, when practiced provides for curing the patient of the viral pathogen so the patient may return to good health.

### BRIEF SUMMARY OF THE INVENTION

With reference to the present invention's conceptual consideration that is centered upon a medical procedural processes, inclusive of its embodiments of medical devices, colloids and gases, the present invention addresses the long-felt need to treat a viral pathogen infecting a patient's pulmonary system. The present invention's inventive leap advantages itself above currently practiced art in the field of endeavor in the medical arts of providing a means to fracture and cleanse a viral pathogen, such as though not limited to, Covid19 and/or SARS-CoV-2 VUI 202012/01 and/or 11.1.1.7 from a patient's pulmonary system. The present invention does so by providing a Microcrystalline Castile Colloid (MCC) formulated from naturally occurring and/or molecularly engineered enzymes that are suspend with a continuous aqueous solution and are modestly molecularly attracted, as hydrophobic, to the lining of a pulmonary system and highly attracted to a lipid membrane. As lipophilic, to a viral pathogen. When introduced through an ELT (nPk) by a Medical Professional (MP) into the lungs of patient who has been tested positive for the aforementioned viral pathogens, the MCC's lipophilic tail coalesces with the lipid membrane of the viral pathogen and fractures the viral pathogen's lipid membrane (bilayer) and whereby the RNA inner contents of the viral pathogen are dispensed into the MCC and are rendered inert and subsequently, by a process of the present invention, purged from the pulmonary system of the patient. And whereby, in a subsequent test performed by a MP the patient test negative for the viral pathogen.

### BRIEF DESCRIPTION OF THE SEVERAL VIEWS OF THE DRAWING(S)

FIG. 1 illustrates a Medical Professional, (MP) wearing a personal protective equipment (PPE).

FIG. 2 illustrates a patient infected with a viral pathogen.

FIG. 3 illustrates a positive test for a viral pathogen.

FIG. 4 illustrates a negative test for a viral pathogen.

FIG. 5 illustrates a volume metric of Microcrystalline Castile Colloid, (MCC).

FIG. 6 illustrates a volume metric of a MCC suspended above and in proximity to the patient utilizing a medical device.

FIG. 7 illustrates a (bi-laterally symmetrical—dual) ELT (nPk) endotracheal lumen tube with a mask and a “Y” branch.

FIG. 8 illustrates a detail of FIG. (7) of a “Y” branch of a bi-laterally symmetrical—dual conduit channel ELT (nPk) endotracheal lumen tube with a “Y” branch and (8a) a butterfly valve in an ‘open’ position, and (8b) a butterfly valve in a ‘closed’ position. Note: all conduit channels may contain butterfly valves, though for legibility purposes, a singular illustration in FIG. 8 of the butterfly valves is provided.

FIG. 9 illustrates a detail of FIG. (7) of a “Y” bi-laterally symmetrical—quadruple conduit channel ELT (nPk)endotracheal lumen tube “Y” branch.

FIG. 10 illustrates a detail of FIG. (9) of four (4) permutations of conduit channels with quantities of 2, 4, 6 and 8.

FIG. 11 illustrates a detail of FIG. (9) of four (4) permutations of “Y” branches with quantities of 2, 4, 6 and 8.

FIG. 12 illustrates a pneumatic nebulizer medical device.

FIG. 13 illustrates a volume metric of medical grade oxygen.

FIG. 14 illustrates a volume metric of medical grade oxygen tank.

FIG. 15 illustrates a medical device of a fluid warmer.

FIG. 16 illustrates a medical device of a mechanized ventilator.

FIG. 17 illustrates a volume metric of an anti-inflammatory fluid.

FIG. 18 illustrates a medical theater.

FIG. 19 illustrates a medical device of a medical theater bed.

FIG. 20 illustrates a medical device of a vacuum.

FIG. 21 illustrates a medical device of a fluid receptacle.

FIG. 22 illustrates a medical device of a tube clamp.

FIG. 23 illustrates a detail of FIG. (7) of a medical device of ELT (nPk) lumen tube mask

FIG. 24 illustrates a medical device of a mechanized percussion machine.

FIG. 25 illustrates a medical device of a section of tubing for transportation of medical fluids and gases.

FIG. 26 illustrates a computerized device programmed with medical software and connected to a network.

FIG. 27 illustrates a network containing a plurality of computerized devices programmed with medical software.

FIG. 28 illustrates a detail of FIG. (26) of a computerized device programmed with medical software and connected to a network

FIG. 29 illustrates a central computer connected to a network.

FIG. 30 illustrates a MP wearing a PPE and patient in a medical theater, the patient being on a medical theater med in the Trendelenburg position and equipped with an operative percussion vest that is connected to a mechanized percussion machine and/or the MP is performing manual percussions of the chest of the patient, and the expression MD/S (nPk) x ELT (nPk) designating a selection of medical solutions in combination with a selection of medical devices chosen by the MP for the purpose of performing the medical procedure, (FLiM).

FIG. 31 illustrates a single molecule of Microcrystalline Castille Colloid (MCC) with a modestly hydrophilic head and a highly lipophilic tail exhibited in natural and/or molecularly engineered occurrence.

FIG. 32 illustrates a viral pathogen, comprised of a least Covid19 and the viral pathogen's lipid membrane.

FIG. 33 illustrates a detail of a micelle within a MCC and a plurality of MCC molecules surrounding a viral pathogen.

FIG. 34 illustrates a viral pathogen, comprised of a least Covid19 and the viral pathogen's lipid membrane and a mucous lining the patient pulmonary system and a singular molecule of an MCC exhibiting a modest molecular attraction to the mucus lining of the patient's pulmonary system and high molecular attraction to the lipid membrane of the viral pathogen.

FIG. 35 illustrates the viral pathogen's lipid membrane being fractured by a single molecule of the MCC and the viral pathogen's RNA being dispensed into a continuous aqueous solution and the viral pathogen being rendered inert.

FIG. 36 illustrates a strand of RNA of the viral pathogen.

FIG. 37 illustrates an expression of a combination of medical fluids and gases with medical devices.

### DETAILED DESCRIPTION OF THE SEVERAL VIEWS OF THE DRAWING(S)

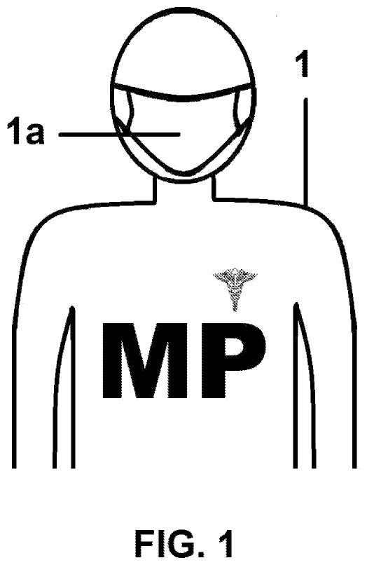

FIG. 1 illustrates a Medical Professional, (MP) (1) wearing a personal protective equipment (PPE) (1a).

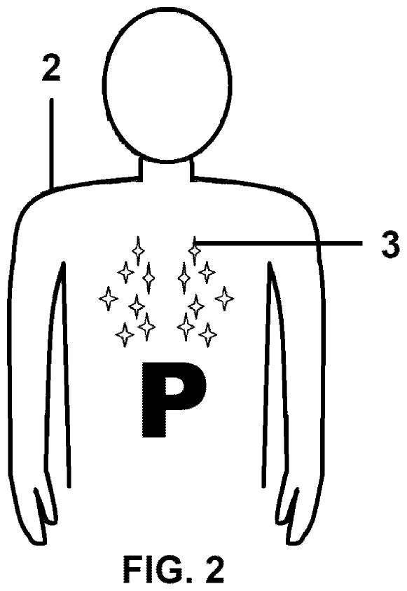

FIG. 2 illustrates a patient (2) infected with a viral pathogen (32), whereas a MP (1) has tested the patient (2) for a viral pathogen (32) and the test result is positive (3).

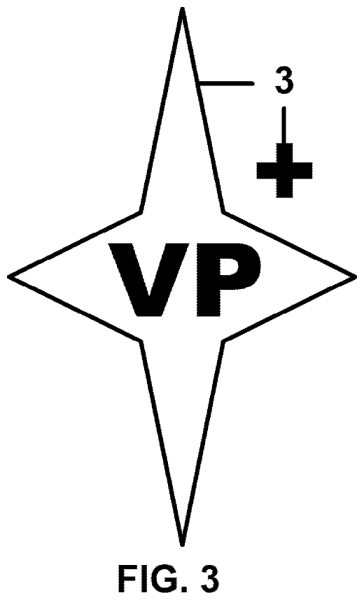

FIG. 3 illustrates a positive test for a viral pathogen, whereas a MP (1) has tested the patient (2) for a viral pathogen (32) and the test result is negative (4).

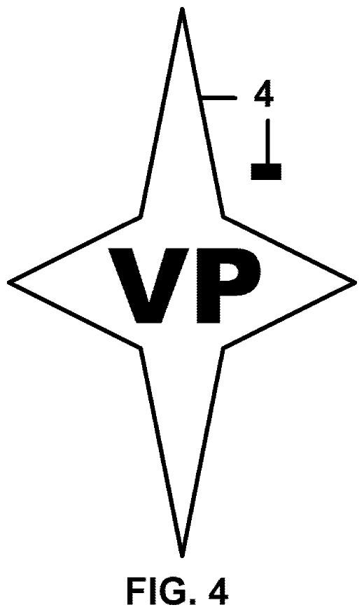

FIG. 4 illustrates a negative test for a viral pathogen (4).

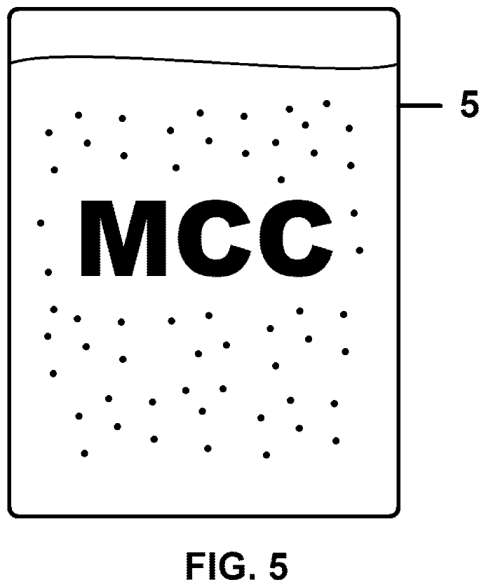

FIG. 5 illustrates a volume metric of Microcrystalline Castile Colloid, (MCC) (5).

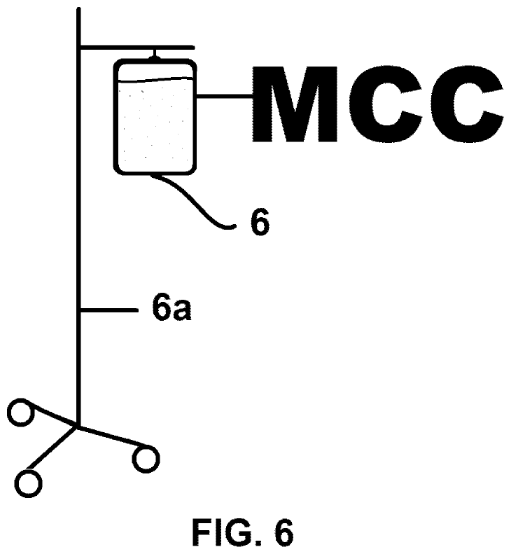

FIG. 6 illustrates a volume metric of a MCC (5) suspended above and in proximity to the patient (2) utilizing a medical device (6a).

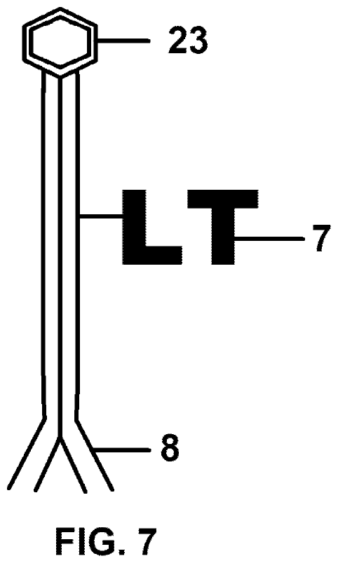

FIG. 7 illustrates a (bi-laterally symmetrical—dual) MD/S (nPk) x ELT (nPk) endotracheal lumen tube with a mask and a “Y” branch.

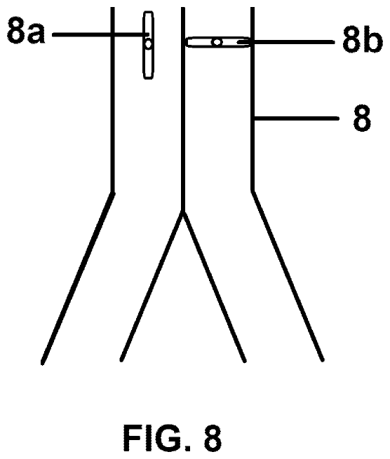

FIG. 8 illustrates a detail of FIG. (7) of a “Y” branch of a bi-laterally symmetrical—dual conduit channel MD/S (nPk) x ELT (nPk) endotracheal lumen tube with a “Y” branch and (8a) a butterfly valve in an ‘open’ position, and (8b) a butterfly valve in a ‘closed’ position. Note: all conduit channels may contain butterfly valves, though for legibility purposes, a singular illustration in FIG. 8 of the butterfly valves is provided.

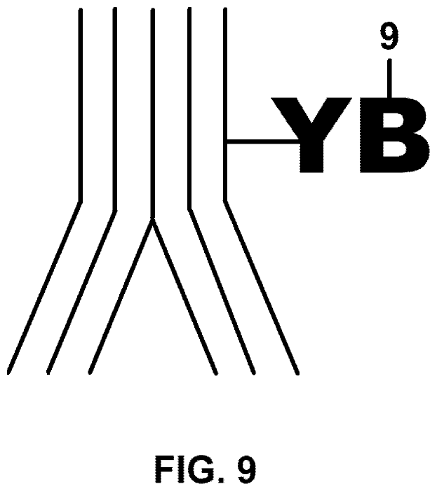

FIG. 9 illustrates a detail of FIG. (7) of a “Y” bi-laterally symmetrical—quadruple conduit channel MD/S (nPk) x ELT (nPk) endotracheal lumen tube “Y” branch.

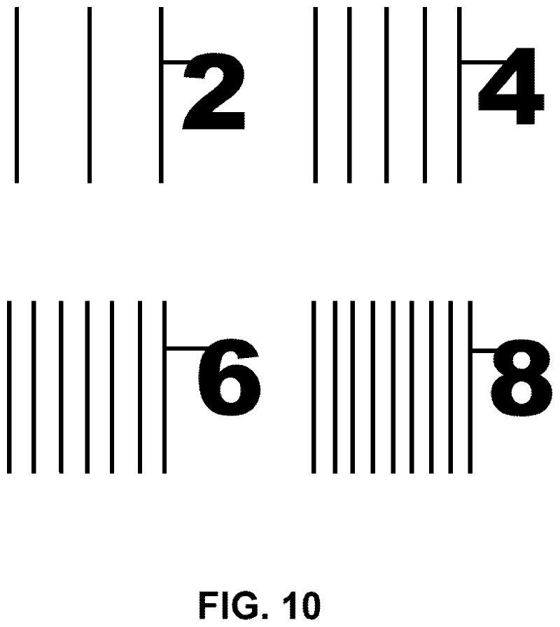

FIG. 10 illustrates a detail of FIG. (9) of four (4) permutations of conduit channels with quantities of 2, 4, 6 and 8.

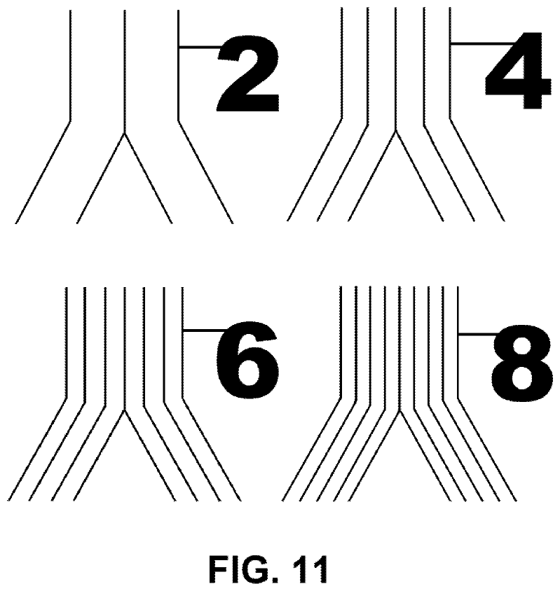

FIG. 11 illustrates a detail of FIG. (9) of four (4) permutations of “Y” branches with quantities of 2, 4, 6 and 8.

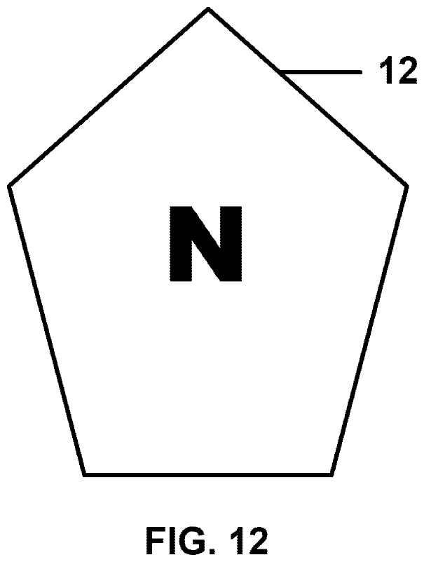

FIG. 12 illustrates a pneumatic nebulizer medical device (12) that converts liquids into a fine mist.

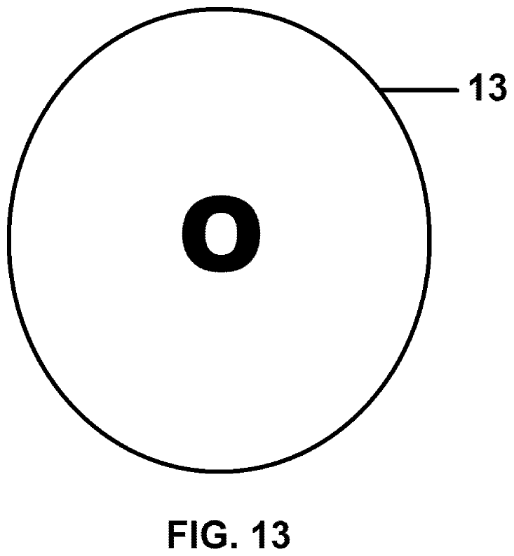

FIG. 13 illustrates a volume metric of medical grade oxygen (13) with deactivated (if initially present) microorganisms such as fungi, bacteria, viruses, spores, unicellular eukaryotic organisms.

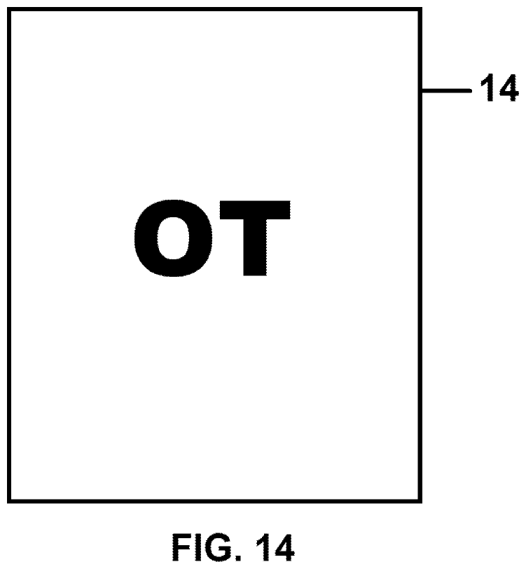

FIG. 14 illustrates a volume metric of medical grade oxygen tank (14) containing deactivated (if initially present) microorganisms such as fungi, bacteria, viruses, spores, unicellular eukaryotic organisms.

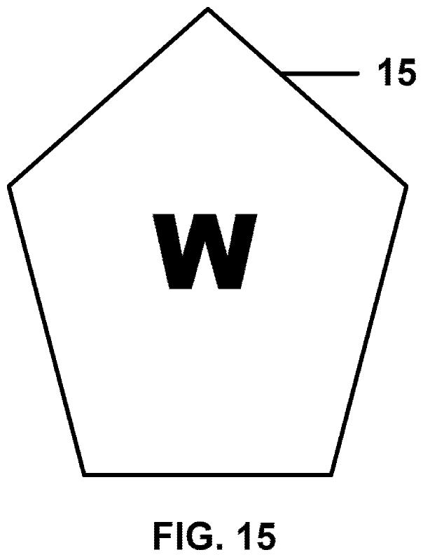

FIG. 15 illustrates a medical device of a fluid warmer (15) that warms medical fluids.

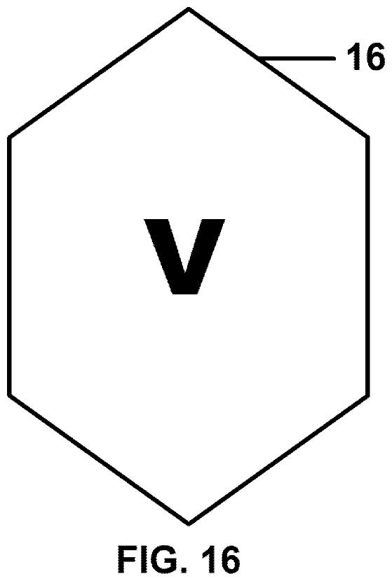

FIG. 16 illustrates a medical device of a mechanized ventilator (16) that provides mechanical ventilation by supplying breathable air into and out of the pulmonary system of the patient (2).

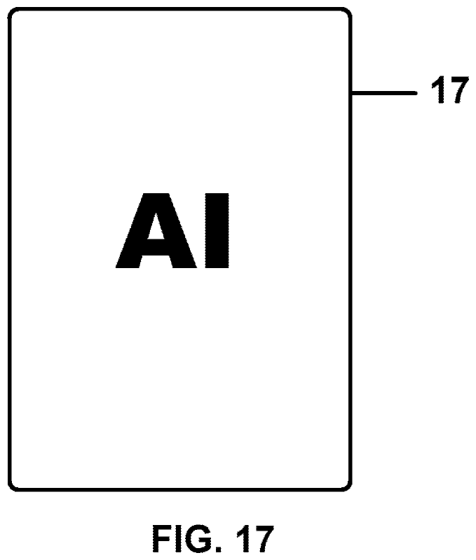

FIG. 17 illustrates a volume metric of an anti-inflammatory fluid (17) containing properties of substances for treatment that reduces inflammation or swelling of the linings of the pulmonary system of the patient (2).

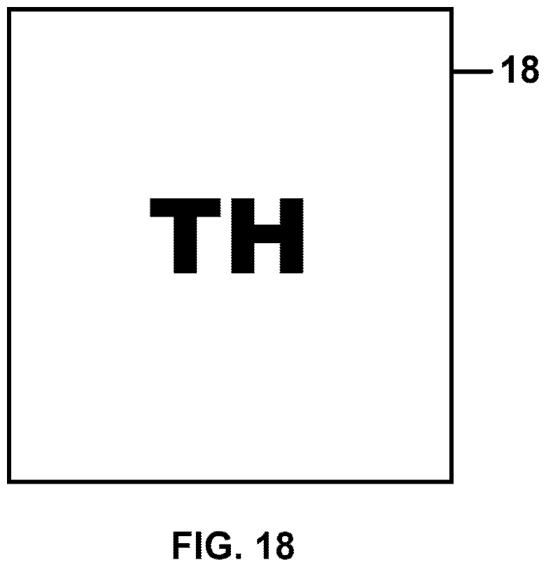

FIG. 18 illustrates a medical theater (18) for performing a process of the present invention in an aseptic environment.

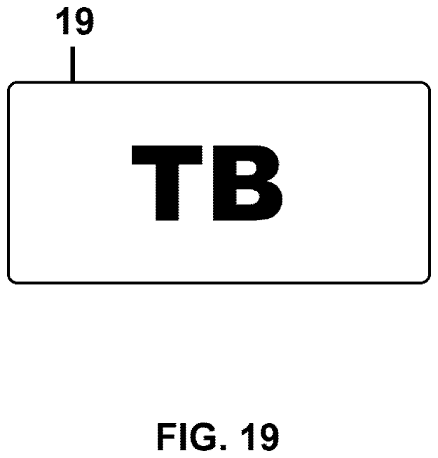

FIG. 19 illustrates a medical device of a medical theater bed (19) for position the patient in a Trendelenburg position.

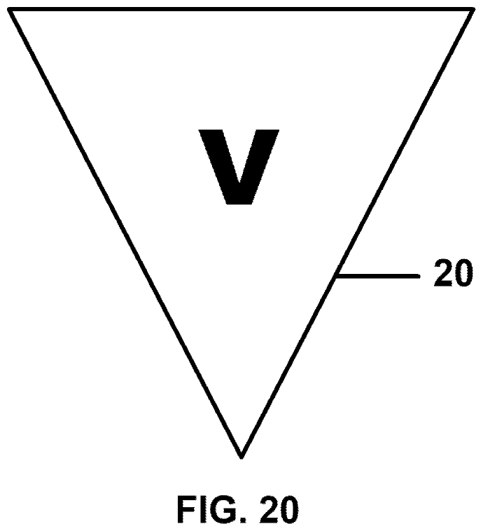

FIG. 20 illustrates a medical device of a vacuum (20) for vacuuming fluids and gases from the pulmonary system of the patient (2).

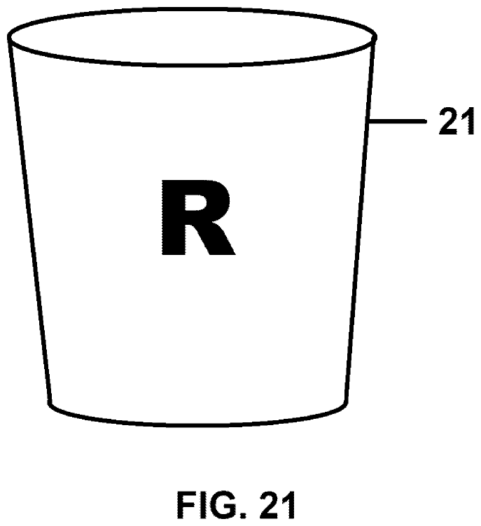

FIG. 21 illustrates a medical device of a fluid receptacle (21) for collecting vacuumed fluids and gases from the pulmonary system of the patient (2).

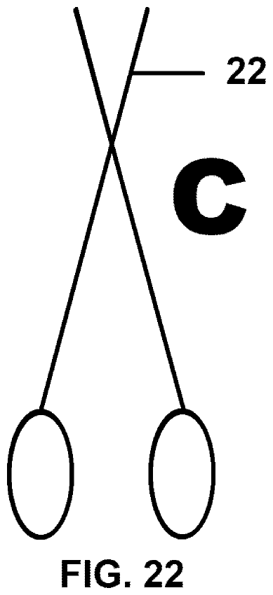

FIG. 22 illustrates a medical device of a tube clamp (22) utilizing as necessitated during the medical procedural process of the present invention utilized or hold or secure objects tightly together to prevent movement or separation and to contain pressurization with a tube (25).

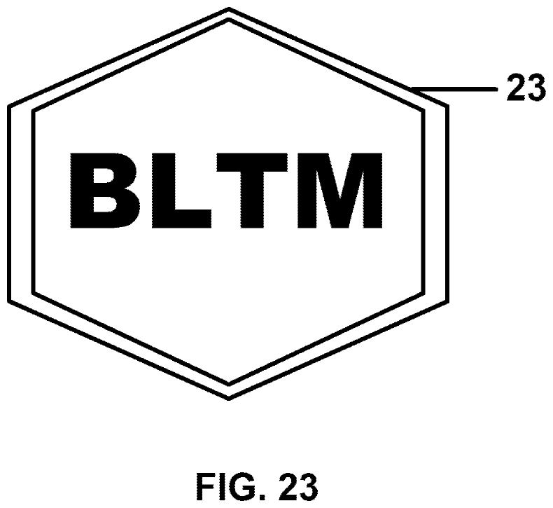

FIG. 23 illustrates a detail of FIG. (7) of a medical device of MD/S (nPk) x ELT (nPk) endotracheal lumen tube mask

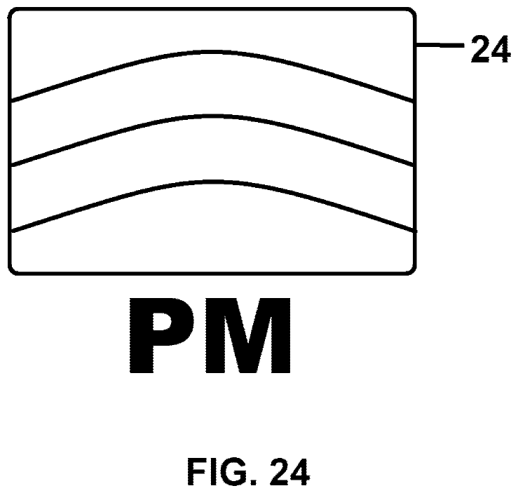

FIG. 24 illustrates a medical device of a mechanized percussion machine (24) utilized in connection with a chest vest for provided kinetic energy in the form of rapidly vibrating the patient's (2) chest for the purpose of mixing and stirring the MCC (5) within the lungs of the patient (2).

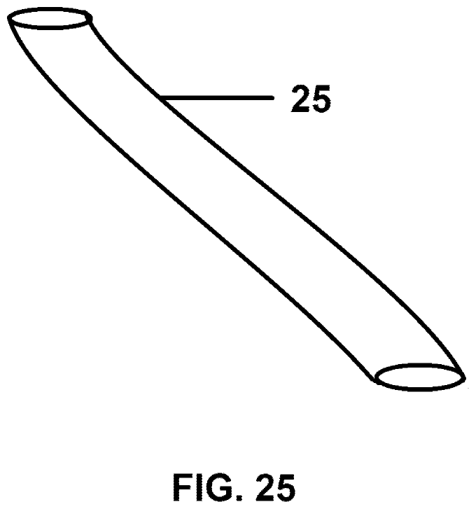

FIG. 25 illustrates a medical device of a section of tubing (25) for transportation of medical fluids and gases.

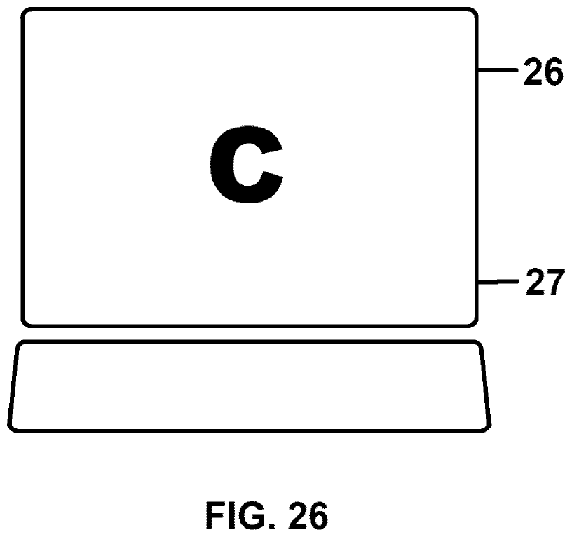

FIG. 26 illustrates a computerized device (26) programmed with medical software and connected to a network (27).

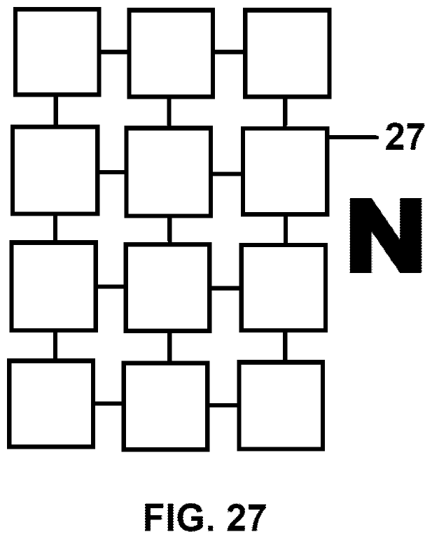

FIG. 27 illustrates a network (27) containing a plurality of computerized devices programmed with medical software.

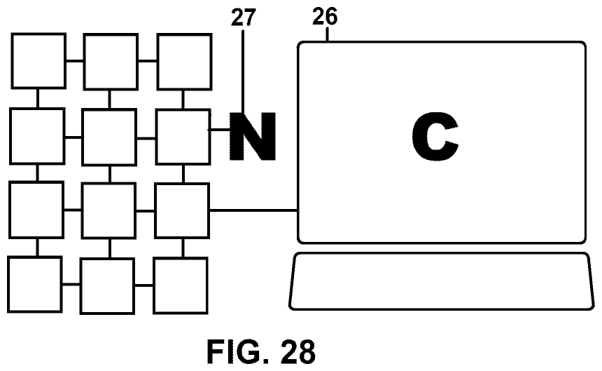

FIG. 28 illustrates a detail of FIG. (26) of a computerized device programmed with medical software and connected to a network (27).

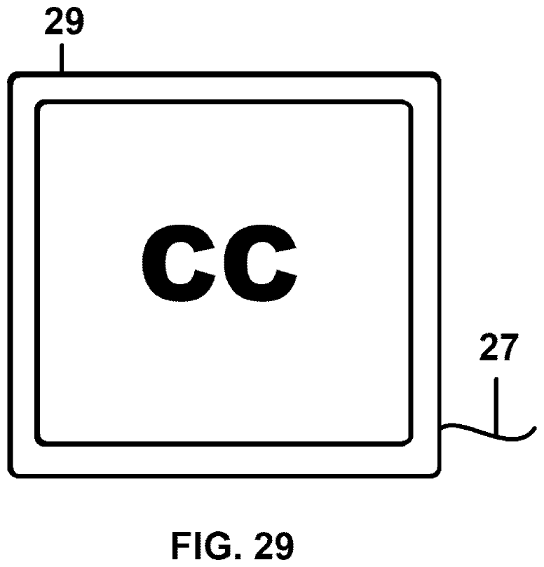

FIG. 29 illustrates a central computer (29) connected to a network (27).

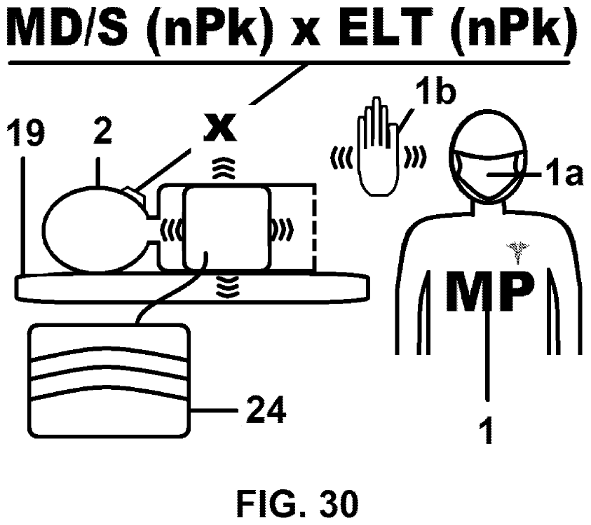

FIG. 30 illustrates a MP (1) wearing a PPE (1a) and patient (2) in a medical theater (18), the patient (2) being on a medical theater bed (19) in the Trendelenburg position and equipped with an operative percussion vest (24) that is connected to a mechanized percussion machine (24) and/or the MP (1) is performing manual percussions (1b) on the chest of the patient (2), and the expression MD/S (nPk) x ELT (nPk) designating a selection of medical solutions in combination with a selection of medical devices chosen by the 1VIP (1) for the purpose of performing the medical procedure, (FLiM).

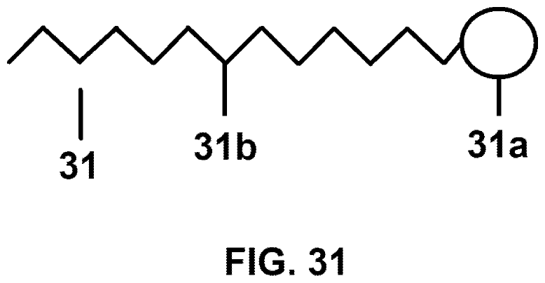

FIG. 31 illustrates a single molecule of Microcrystalline Castille Colloid (MCC) (31) with a modestly hydrophilic head (31a) and a highly lipophilic tail (31b) exhibited in natural and/or molecularly engineered occurrence.

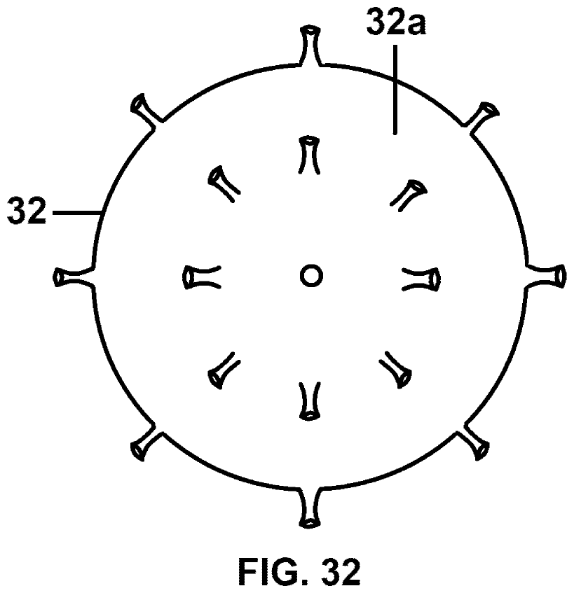

FIG. 32 illustrates a viral pathogen (32), comprised of a least Covid19 and the viral pathogen's (32) lipid membrane (32a).

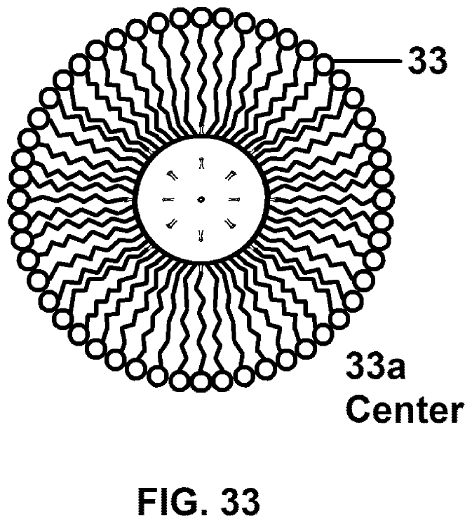

FIG. 33 illustrates a detail of a micelle (33) within a MCC (5) and a plurality of MCC (5) molecules surrounding a viral pathogen (32).

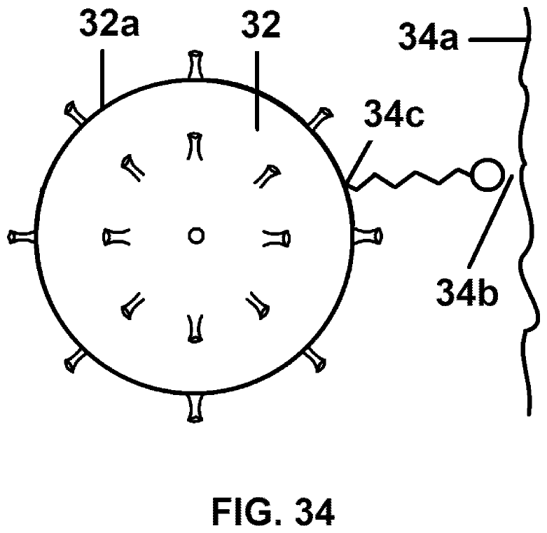

FIG. 34 illustrates a viral pathogen (32), comprised of a least Covid19 and the viral pathogen's (32) lipid membrane (32a) and a mucous lining the patient pulmonary system (34a) and a singular molecule of an MCC (5) exhibiting a modest molecular attraction (34b) to the mucus lining of the patient's pulmonary system (34a) and high molecular attraction (34c) to the lipid membrane of the viral pathogen (32a).

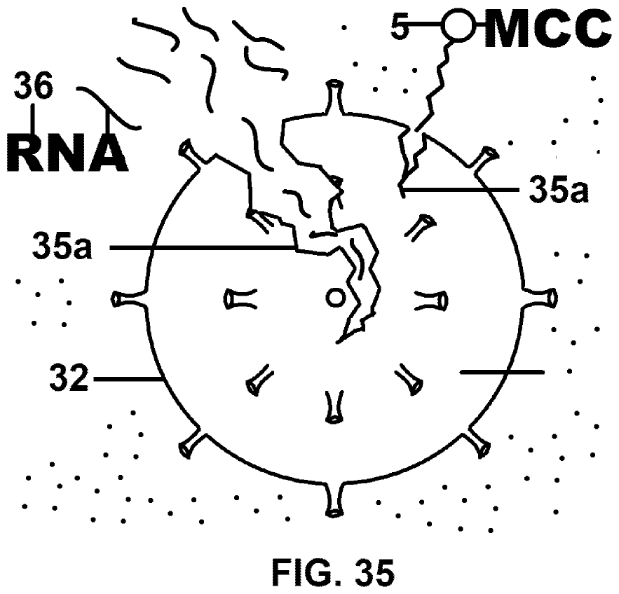

FIG. 35 illustrates the viral pathogen's lipid membrane (32a) being fractured (35a) by at least a single molecule of the MCC (5) and the viral pathogen's RNA (36) being dispensed into a continuous aqueous solution and the viral pathogen (32) being rendered inert.

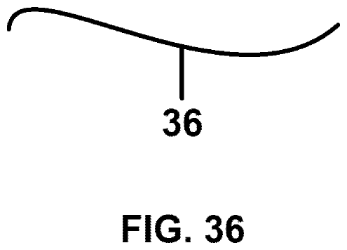

FIG. 36 illustrates a singular strand of RNA (36) of the viral pathogen (32).

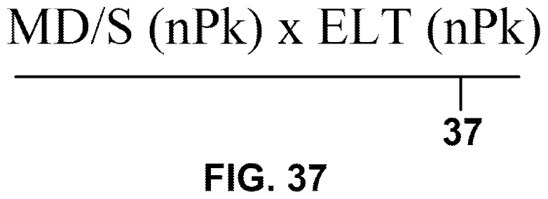

FIG. 37 illustrates an expression of a combination of medical fluids and gases with medical devices as: MD/S (nPk) x ELT (nPk).

### DETAILED DESCRIPTION OF THE INVENTION

Ternary phased medical procedural process for fracturing a lipid membrane, (bilayer) of a viral pathogen and cleansing the viral pathogen from a patient's pulmonary system, (FLiM), of an individual, herein known as “patient” or “the patient”, by a ternary phased medical procedural performed by a Medical Professional (MP), whereas, in one embodiment the FLiM consists of at least; reviewing a medical history of the patient, diagnosing a patient as positive for a viral pathogen, determining that the viral pathogen is encapsulated by a lipid membrane, determining the viral pathogen is infecting a pulmonary system of the patient, selecting a medical device of the medical procedure, prepping the patient for the medical procedure, performing a procedure upon the patient, diagnosing the patient as negative for a viral pathogen, rehabilitating the patient and following-up post procedure the patient's status; comprised of: at least one (1) continuous aqueous suspension, at least (1) medical device, at least one (1) medical theater, at least one (1) central computer, at least one (1) computerized device, at least one (1) network, and whereas: a) reviewing, by a 1VIP, a medical history of the patient's medical case history, or anamnesis, more particularly viral infections, treatment and medication, obtained by the MP through definitive inquires, either of the patient or of others who know the patient for a purposes of attaining supplemental information pertinent a focused diagnosis of the patient; b) diagnosing, by a MP, as a result of conducting a medical examination upon the patient, the patient's infectious status as positive for a viral pathogen the diagnosis determining that the patient is positive for a viral pathogen, for example, Covid19, and the patient's symptoms and/or condition is consistent with the diagnosis; c) diagnosing, by a MP as a result of conducting a medical examination, that the viral pathogen is infecting the patient's pulmonary system by review of an x-ray and whereby a result of the x-ray contains indications of findings of abnormalities with peripheral Ground Glass Opacities, (GGO) affecting the lower lobes; d) determining, by a MP as a result of conducting a medical examination, ribonucleic acids (RNA) of the viral pathogen are encapsulated by a lipid membrane, whereby in the instance of a test for COVID-19 the testing involves analyzing samples to assess the current or past presence of SARS-CoV-2 and upon test analyzation a finding that the two main branches detect either the presence of the virus or of antibodies produced in response to infection; e) determining, by a MP, a medical procedure to eliminate the viral pathogen from the pulmonary system of the patient such as a FLiM process as illustrated herein; t) determining, by a MP, a causative order of the steps of the medical procedure, for example, an election of steps for inclusion in the medical process is determined or are arranged in view of a test result and symptom of the patient and how the occurrence of some steps may affect other steps, while other steps may not influence one another; g) determining, by a MP, from a diagnostic reading a volume metric concentration of an MCC, an air flow rate, an interval, a pressurization, a manual or machine percussion duration, a utilization of a medical device(s), a volume metric of a suspension(s), and a duration of the medical procedure that a equal to or relatively equal to an election of steps in MD/S (nPk) x ELT (nPk) for a purpose of providing a tailored treatment to the patient; h) programming, by a MP, a computerized device to process a medical data of the medical procedure whereby is dually trained MP in the fields of endeavor of the medical and technical arts utilizes a preexisting computer program, designs or builds an executable computer program to accomplish the specific computing tasks in relation to the FLiM, and whereas, i) equipping, by a MP, a medical theater with network connectivity means, a Central Computer equipped with a Non-Transitory Computer Readable Medium Database, (CCNTCRMD) a computerized device and connecting the central computer to the network that is as well performed by a dually trained MP in the fields of endeavor of the medical and technical arts in order to accomplish the specific computing tasks in relation to the FLiM.

And whereas, (2) a process of claim 1 further comprising the steps of; a) performing, by a 1VIP, a first phase of the ternary phased medical procedure in performing a lung(s) lavage procedure upon the patient where the 1VIP prepares, initially and generally, the patient for the FLiM; b) prepping, by a MP, the patient pre-medical procedure by providing the patient with a series of information and instructions that advise the patient of the nature and intention of the medical procedure, briefing the patient about how the medical procedure is performed, advising the patient of patient's responsibilities, pre, during and post procedure, and providing the patient with and collecting the patient's signature upon a contractual agreement; c) prepping, by a MP, the patient for a first step of the ternary phased medical procedure, by sanitizing, dressing, delivering the patient to a medical theater, and initially positioning the patient in a horizontal supine position upon an operating table in order to perform a whole, (bilateral) lung(s) lavage (WLL) in the present invention also understood as FLiM and whereas such terms may and are used utilized interchangeably; d) anesthetizing, by a MP, generally, the patient for the medical procedure, FLiM, where a MP induces medical coma and whereas the patient losses protective reflexes, consequential to the administration of at least one general anesthetic agent administered by the MP, e) preoxygenating, by a MP, before anesthetic induction and tracheal intubation a lung of the patient where the MP preoxygenates the patient's lungs with a tightly fitting oxygen mask that is connected to an external oxygen containing vessel, f) prepping by a MP, the patient for a first step of the ternary phased medical procedure, for a purpose of initially cleansing the lung of suptum, and excessive surfactants, secretion and residual involvement by utilization of a machine and/or medical device and/or manual percussion and the introduction of a vaporous cleansing agent through the patient trachea and the utilization of a medical device such as a pulmonary vacuum that is applied by the MP and induces a lower atmospheric pressurization resulting in the suptum, and excessive surfactants, secretion and residual involvement being withdrawn from the patient's pulmonary system and into an external receptacle, g) obtaining, by a MP, a pre WLL estimation of the patient's baseline Functional Residual Capacity, (FRC) whereas a MP by means of a medical device, gauges a volume metric of air present in a lung(s) at the conclusion of passive expiration. and whereas a MP gauges Shunt Fraction, (SF) of a passage of deoxygenated blood from the right side of the patient's heart to the left side of the patient's heart without participation in gas exchange in the pulmonary capillaries, h) preparing, by a MP, a reservoir volume of a saline solution and suspending the saline solution reservoir volume in proximity to and above the patient, whereas a MP prepares a saline solution as a mixture of sodium chloride with a continuous aqueous solution and suspends the solution in proximity to the patient utilizing a medical device and such devices having means to interconnect to a secondary device of the present invention in order to deliver the saline solution to the patient or an independent tubular connection that has means to deliver the saline solution to the patient i) determining, by a MP, through imaging and/or V/Q scan, a lung of the patient with a greater viral infectious status, secretions and residual involvement, considering a lung with a greater viral infectious status, secretions and residual involvement as a primary target lung, preserving the patient in a horizontal supine position or re-positioning the patient in a Trendelenburg position upon the operating table that elevates a side of the patient containing the primary target lung, whereas a MP utilizes a medical imaging device and isotope, such a scintigraphy and medical isotopes to evaluate circulation of air and blood within the patient's lungs and then places the patient in a flat and inclining, declining or horizontally parallel Trendelenburg position upon the operating table as determined by a MP for a purpose of performing a medical procedure upon the patient, j) placing, by a MP, upon the chest of the patient, a vest for chest physiotherapy whereas a MP wraps the patient's upper torso in a close-fitting, shoulder to waist-length, sleeveless vest that is connected to a secondary medical device for a purpose of performing machine percussion upon the chest of the patient during the medical procedure k) intubating, by a MP, the patient with a MD/S (nPk) x ELT (nPk) endotracheal lumen tube; whereas a MP inserts a flexible plastic MD/S (nPk) x ELT (nPk) tube into the trachea of the patient for a purpose of maintaining an open airway and/or to function as a conduit through which to dispense a solution(s) into a lung(s) of the patient during the medical procedure l) performing, by a MP, a bronchoscopy upon the patient, and ensuring a correct placement of the MD/S (nPk) x ELT (nPk)) endotracheal lumen tube into the primary target lung of the patient whereas a MP inserts a bronchoscope into through the MD/S (nPk) x ELT (nPk) endotracheal lumen tube conduit for a purpose of visualizing the inside of the patient's lungs for a purpose of ensuring a correct placement of the MD/S (nPk) x ELT (nPk) endotracheal lumen tube into the primary target lung of the patient, m) isolating, by a MP, each lung of the patient by means of pressurizing, by a MP, each lung of the patient with oxygen by means of a ventilator and examining each lung for oxygen leakage by means of venting the non-ventilated lung endotracheal tube orifice into a saline water sealed reservoir concurrently as the ventilated lung is maintained at an airway guided preset air pressurization and inspecting by a MP the saline water reservoir for froth; n) determining, by a MP, the patient's general preparedness for a medical procedure, WLL, whereas a MP generally evaluates a medical device reading and visually and manually inspects the patient's general preparedness for the medical procedure, and o) performing, by a MP, a secondary phase of a ternary medical procedure upon the patient whereas a MP performs a secondary phase of a ternary medical procedure upon the patient with reference to a process of the present invention, notably at least one step of a medical process as illustrated in claims 1-3.

A step of claim 2 of performing, by a MP, a secondary phase of a ternary medical procedure upon the patient a medical procedure upon the patient that fractures the lipid membrane encapsulating the viral pathogen that is infecting the patient's pulmonary system, cleansing the fractured lipid membrane and the unencapsulated RNA of the viral pathogen from the patient's pulmonary system and rehabilitating the patient further comprising the steps of, a) determining, by a MP, a vital sign of the patient is in a range and monitoring a vital sign of the patient during the medical procedure; b) introducing, by a MP, through the MD/S (nPk) x ELT (nPk) endotracheal tube and into an elevated side lung cavity of the patient, a continuous aqueous transport medium of a Microcrystalline Castile Colloid (MCC), of a semi-homogeneous amalgam of a modestly hydrophobic and highly lipophilic profile; c) performing, by a MP, manual chest percussions and/or mechanized chest percussion on the chest of the patient for a duration of time consistent with a pre-determined casual order and volume metrics as determined by a step of claim 1; whereas a MP utilizing the hands of the MP claps on the chest and/or back of the patient for the purpose of agitating the MCC inside of the patient's lungs, or a MP utilizes medical device specifically manufactured for chest percussions that disquiets and agitates the MCC inside the patient lungs and so whereas in each instance of chest percussion the MCC inside of the patient's lungs is agitated amongst the viral pathogen infecting the patient lungs and whereby the MCC may through compulsory interaction of the MCC and the viral pathogen, by means of a step of the present invention that is illustrated in claim 1 to achieve efficacy, d) performing, by a MP, a cleansing of the patient's target lung by fracturing the lipid membrane of the viral pathogen and the un-encapsulating the RNA of the viral pathogen of the viral pathogen by utilizing the MCC, chest percussions and pre-determined casual order and volume metrics as pre-determined a MP, e) determining, by a MP, a viral pathogen's lipid membrane is fractured by the MCC, manual chest percussions and/or mechanized chest percussion on the chest of the patient for a duration of time consistent with a pre-determined casual order and volume metrics as determined by a step of claim 1, and its unencapsulated RNA is freely suspended within the MCC, whereas a MP determines the MD/S (nPk) x ELT (nPk) and volumes metrics inclusive of a duration of time and volume metric has achieve efficacy, f) draining, by a MP, the MCC, inclusive of the viral pathogen's unencapsulated RNA, out of the patient's target lung and into an external vessel, whereas a MP utilizing the medical device of a vacuum and the MD/S (nPk) x ELT (nPk) aspirates the patient's lungs utilizing the medical device of a vacuum drains the MCC unencapsulated RNA, out of the patient's target lung and into an external vessel, g) rinsing, by a MP, the MCC, inclusive of the viral pathogen's unencapsulated RNA, out of the patient's target lung and into an external vessel utilizing a continuous aqueous therapeutic solution whereas a MP, by utilization of a rinsing agent, such as a continuous aqueous medium such as clinically sterile water, that may contain therapeutic agent such an anti-inflammatory, and by means of or bronchoalveolar lavage, rinses the patient's lungs for a purpose of excising the MCC and viral pathogen's unencapsulated RNA, out of the patient's target lung and into an external vessel, h) determining, by a MP, utilizing a visualization and/or medical device that an initial volume metric of a MCC in an external vessel contains and acceptable or non-acceptable concentration of unencapsulated RNA of a viral pathogen, whereas a MP visually and/or by utilization of a medical device, determines that an initial volume metric of a MCC in an external vessel contains and acceptable or non-acceptable concentration of unencapsulated RNA of a viral pathogen, i) repeating, by a MP, as necessary, a step of a claim 1, step (h) until a secondary or thereafter volume metric of a MCC in an external vessel contains an acceptable concentration of unencapsulated RNA of a viral pathogen; and whereas j) repositioning, by a MP, the patient on the medical table, and repeating a step of claim 1 and/or claim 2 on a second lung of the patient.

A process of claim 1 of performing, by a MP, a ternary phase of a ternary phased medical procedure upon the patient further comprising the steps of; a) diagnosing, by a MP, as a result of conducting a post-procedural medical examination upon the patient, the patient's infectious status as negative for a viral pathogen whereas a MP, through a diagnosis evaluation by means of a medical test determines the patient's infectious status as negative for a viral pathogen, b) rehabilitating, by a MP, the pulmonary system of the patient, whereas a MP by means of a multidisciplinary, and thorough intervention administers a series of respiratory therapeutic sessions comprising at least respiratory medications, oxygen therapy, exercise techniques, exercise reconditioning sessions and oxygen dosing for a purpose of rehabilitating the patients lungs to a nominal functioning state, c) following-up, by a MP, post medical procedure with the patient, by checking and monitoring a vital sign of the patient, determining as required, a length of stay in a medical facility by a MP, prescribing, as required a medication by a MP, discharging as required, the patient from a medical facility and providing by a MP the patient with a post-procedural recovery instruction to follow, and determining by a MP, as required, a return visit by the patient to test an infectious status of the patient, whereas a MP follow-up with the patient in reference to the patient health status, inclusive of the patient viral pathogen infectious status, and whereas d) recording, by a MP, by utilizing the computer and the network, a result of the FLiM in the CCNTCRM for patient medical recording purposes and for clinical trial evaluation, whereas a MP by utilizing the computer and the network, a result of the FLiM in the CCNTCRM for patient medical recording purposes and for clinical trial evaluation, enters a registry of at least the following in the CCNTCRM: an initial health status of the patient, a determination of a medical procedure, a selection of (x/x), a volume metric of (x/x), a causal order of steps utilized during the medical procedure and post-procedural health status of the patient.

### Sequence Listing. (N/A)

Glossary of Terms

(a) Microcrystalline Castille Colloid, (MCC), ‘a continuous aqueous solution, also understood as a ‘suspension’ or aerosol derivative thereof, comprised of at least a molecular particle (of the MCC) being suspended in a continuous aqueous solution, and being molecularly natural and/or molecularly engineered in occurrence. The molecular particle of the MCC being molecularly natural in occurrence and/or molecularly engineered as modestly hydrophobic and highly lipophilic, whereas, the molecular particle of the MCC being a dual compound, being polymorphic in molecularly natural and/or molecularly engineered occurrence as, ‘amphiphilic’ or ‘amphipathic’ and possessing both hydrophilic and lipophilic properties. The MCC being constituted of two (2), molecular bonded portions, the bonded portions of the MCC being ‘loosely’ bonded.

A first portion of the MCC being a ‘polar head’ of the molecular particle of the MCC, exhibiting an intermolecular force being modestly hydrophobic in the MCC's molecularly natural occurrence and/or molecularly engineered occurrence, and a secondary portion of the MCC being a ‘non-polar hydrocarbon tail ’ of the molecular particle of the MCC and exhibiting an intermolecular force being highly lipophilic in the MCC molecularly natural occurrence and/or molecularly engineered occurrence. The molecular particle of the MCC being substantially evenly dispersed within a continuous aqueous transport medium or aerosol derivative. The continuous aqueous transport medium comprising at least a molecular structure of two (2) atoms of hydrogen, (Standard atomic weight Ar, std [1.00784, 1.00811], Conventional: 1.008), and one (1) atom of oxygen, (Standard atomic weight [15.99903, 15.99977], Conventional: 15.999). A common phraseology of the continuous aqueous transport medium being understood as, though not limited to such understanding as, ‘H²0’ or, ‘water’ or an applicable phraseology within a contextualization of the continuous aqueous transport medium. The continuous aqueous transport medium of the water, being ‘soft’ in composition, and/or deionized.

As the non-polar hydrocarbon tail of the MCC consists of the intermolecular force being modestly hydrophobic in the molecularly natural occurrence and/or molecularly engineered occurrence, and a ‘mucous lining’ of a ‘surface’ of the respiratory system being primarily enveloped by the mucous lining, and the mucous lining being comprised of a substantially aqueous-based gel consisting of glycoproteins, immunoglobulins, lipids and ‘other substances’ the other substances of the mucous lining being immaterial in consequence with reference to a process of the present invention in terms of at least a molecular force and volume. The non-polar hydrocarbon tail of the MCC having an intermolecular force being modestly molecularly attracted to aqueous-based gel of the mucous lining gel consisting of glycoproteins, immunoglobulins, lipids, and other immaterial substances, of the respiratory system. And as the non-polar hydrocarbon tail of the MCC contains the intermolecular force being highly lipophilic in the molecularly natural occurrence and/or molecularly engineered occurrence, the MCC having a tendency to be highly molecularly and more so attracted to a lipid then to an aqueous-based mucous lining of respiratory system.

A viral pathogen, such as though not limited to Covid19, and mutations thereof, SARS-CoV-2 VUI 202012/01 and/or B.1.1.7 being comprised of an encapsulating outer shell (bilayer), the encapsulating outer shell of the viral pathogen being substantially comprised of a lipid. The lipid being substantially insoluble in an aqueous medium, such as though not limited to the mucous lining of the pulmonary system, and the encapsulating outer shell lipid being substantially soluble in molecularly naturally occurring organic solvents and/or substantially soluble in molecularly engineered solvents, such as though not limited to the MCC.

How the MCC fractures the lipid membrane of the viral pathogen.

The MCC, when introduced into a pulmonary of a patient that is infected with the viral pathogen, forms micelles that surround the viral pathogen while attempting to remove itself from the H20 of the continuous aqueous medium that is substantially comprised of H20. And, whereas at least one lipophilic tail of the MCC fractures the lipid membrane of the viral pathogen, by attempting to coalesce with or wedge into the lipid membrane of the viral pathogen, fracturing the lipid membrane (bilayer) of the viral pathogen, and whereas the viral pathogen dispenses its RNA, through its fractured lipid membrane into the continuous aqueous solution and whereas, effectually, the viral pathogen becomes inert.

With reference to the aforementioned, “how the MCC fractures the lipid membrane of the viral pathogen” for clarity purposes, as the present invention comports a plurality of medical devices, processes, solutions and suspensions, and a MP in relation to the medical condition of a patient may select various combinations of medical devices processes, solutions and suspensions it should be understood that a potential of permutations of a process of the present invention arise and are available for the MP to practice. It is therefore an objective to provide a MP with a selection of devices, processes, solutions, and suspensions and as such, a combination in terms of permutations of devices, processes, solutions, and suspensions. For legibility purposes, when a such permutations are made available to a MP during a process of the present invention the following expression is utilized in order to illustrate the potential permutations: “MD/S (nPk) x ELT (nPk)”, whereby “MD” represents Medical Devices, “S” represents Solution and/or Suspensions, “x” represent “in combination” and ELT represents Endotracheal Lumen Tube.

The foregoing description conveys the soundest understanding of the objectives, advantages, and inventive leap of the present invention. Diverse embodiments may be formed from the inventive concept of the present invention. It is to be understood that matter disclosed herein is to be interpreted not as in a limiting sense, though rather as illustrative. For the purpose of this disclosure, like reference numerals in the figures shall refer to like features unless otherwise indicated or is obvious by context. The subject device and method of use is sometimes referred to as the device, the invention, the process, the technology, the machine, or other similar terms. These terms may be used interchangeably as context requires and from use the intent becomes apparent. The plural may include the singular and singular the plural as appropriate from a fair and reasonable interpretation of a condition of a phraseology. “The” or “a” medical professional (MP) may be referred to as “the” or “a” MP, doctor, practitioner or like term in the field of the medial arts. The individual may be referred to as “the” or “a” patient, or like term in the field of the medial arts. The illustrations (drawings) as well comport a similar understand whereby the drawings are intended to be interpreted not as in a limiting sense, though rather as illustrative. The specification, inclusive of a molecular structure of a solution and/or suspension contained within the solution or standing along is intended to be interpreted not as in a limiting sense, though rather as illustrative, as well as a process, medical devices, computerized machines and network illustrated in the present invention. The abstract contains the range and scope of the present invention and is to be interpreted not as in a limiting sense, though rather as illustrative. As well, the claims set forth a conceptual consideration of the present invention and are to be interpreted not as in a limiting sense, though rather as illustrative of a utility of the present invention in the field of endeavor to which the present invention pertains.

1) Ternary phased medical procedural process for fracturing and cleansing a viral pathogen from a pulmonary system of an individual, herein known as “the patient” or “patient”, by a ternary phased medical procedural performed by a Medical Professional (MP), consisting of at least; reviewing a medical history of the patient, diagnosing a patient as positive for a viral pathogen, determining that the viral pathogen is encapsulated by a lipid membrane, determining the viral pathogen is infecting a pulmonary system of the patient, selecting a medical device of the medical procedure, prepping the patient for the medical procedure, performing a procedure upon the patient, diagnosing the patient as negative for a viral pathogen, rehabilitating the patient and following-up post procedure the patient's status; comprised of: at least one (1) continuous aqueous suspension, at least (1) medical device, at least one (1) medical theater, at least one (1) central computer, at least one (1) computerized device, at least one (1) network and comprised of at least the steps of:
a) reviewing, by a MP, a medical history of the patient;
b) diagnosing, by a MP, as a result of conducting a medical examination upon the patient, the patient's infectious status as positive for a viral pathogen;
c) diagnosing, by a MP as a result of conducting a medical examination, that the viral pathogen is infecting the patient's pulmonary system;
d) determining, by a MP as a result of conducting a medical examination, that ribonucleic acids (RNA) of the viral pathogen are encapsulated by a lipid membrane;
e) determining, by a MP, a medical procedure to eliminate the viral pathogen from the pulmonary system of the patient;
f) determining, by a MP, a causative order of the steps of the medical procedure;
g) determining, by a MP, from a diagnostic reading a volume metric concentration of an MCC, an air flow rate, an interval, a pressurization, a manual or machine percussion duration, a utilization of a medical device(s), a volume metric of a suspension(s), and a duration of the medical procedure;
h) programming, by a MP, a computerized device to process a medical data of the medical procedure; and
i) equipping, by a MP, a medical theater with network connectivity means, a Central Computer equipped with a Non-Transitory Computer Readable Medium Database, (CCNTCRMD) a computerized device and connecting the central computer to the network.

2) A process of claim 1 further comprising the steps of;
a) performing, by a MP, a first phase of the ternary phased medical procedure in performing a lung(s) lavage procedure upon the patient;
b) prepping, by a MP, the patient pre-medical procedure by providing the patient with a series of information and instructions that advise the patient of the nature and intention of the medical procedure, briefing the patient about how the medical procedure is performed, advising the patient of patient's responsibilities, pre, during and post procedure, and providing the patient with and collecting the patient's signature upon a contractual agreement;
c) prepping, by a MP, the patient for a first step of the ternary phased medical procedure, by sanitizing, dressing, delivering the patient to a medical theater, and initially positioning the patient in a horizontal supine position upon an operating table in order to perform a whole, (bilateral) lung(s) lavage (WLL);
d) anesthetizing, by a MP, generally, the patient for a medical procedure, WLL;
e) preoxygenating, by a MP, before anesthetic induction and tracheal intubation a lung of the patient;
f) prepping by a MP, the patient for a first step of the ternary phased medical procedure, for a purpose of initially cleansing the lung of suptum, and excessive surfactants, secretions, and residual involvement;
g) obtaining, by a MP, a pre WLL estimation of the patient's baseline Functional Residual Capacity, (FRC) and Shunt Fraction, (SF);
h) preparing, by a MP, a reservoir volume of a saline solution and suspending the saline solution reservoir volume in proximity to and above the patient;
i) determining, by a MP, through imaging and/or V/Q scan, a lung of the patient with a greater viral infectious status, secretions and residual involvement, considering a lung with a greater viral infectious status, secretions and residual involvement as a primary target lung, preserving the patient in a horizontal supine position or re-positioning the patient in a Trendelenburg position upon the operating table that elevates a side of the patient containing the primary target lung;
j) placing, by a MP, upon the chest of the patient, a vest for chest physiotherapy;
k) intubating, by a MP, the patient with a MD/S (nPk) x ELT (nPk) endotracheal lumen tube;
l) performing, by a MP, a bronchoscopy upon the patient, and ensuring a correct placement of the MD/S (nPk) x ELT (nPk) endotracheal lumen tube into the primary target lung of the patient;
m) isolating, by a MP, each lung of the patient by means of pressurizing, by a MP, each lung of the patient with oxygen by means of a ventilator and examining each lung for oxygen leakage by means of venting the non-ventilated lung endotracheal tube orifice into a saline water sealed reservoir concurrently as the ventilated lung is maintained at an airway guided preset air pressurization and inspecting by a MP the saline water reservoir for froth;
n) determining, by a MP, the patient's general preparedness for a medical procedure, WLL; and
o) performing, by a MP, a secondary phase of a ternary medical procedure upon the patient.

3) A step of claim 1 of performing, by a MP, a secondary phase of a ternary medical procedure upon the patient a medical procedure upon the patient that fractures the lipid membrane encapsulating the viral pathogen that is infecting the patient's pulmonary system, cleansing the fractured lipid membrane and the unencapsulated RNA of the viral pathogen from the patient's pulmonary system and rehabilitating the patient further comprising the steps of;
a) determining, by a MP, a vital sign of the patient is in a range and monitoring a vital sign of the patient during the medical procedure;
b) introducing, by a MP, through the MD/S (nPk) x ELT (nPk) endotracheal lumen and into an elevated side lung cavity of the patient, a continuous aqueous transport medium of a Microcrystalline Castile Colloid (MCC), of a semi-homogeneous amalgam of a modestly hydrophobic and highly lipophilic profile;
c) performing, by a MP, manual chest percussions and/or mechanized chest percussion on the chest of the patient for a duration of time consistent with a pre-determined casual order and volume metrics as determined by a step of claim 1;
d) performing, by a MP, a cleansing of the patient's target lung by fracturing the lipid membrane of the viral pathogen and the un-encapsulating the RNA of the viral pathogen of the viral pathogen by utilizing the MCC, chest percussions and pre-determined casual order and volume metrics as pre-determined a MP;
e) determining, by a MP, a viral pathogen lipid's membrane is fractured by the MCC, manual chest percussions and/or mechanized chest percussion on the chest of the patient for a duration of time consistent with a pre-determined casual order and volume metrics as determined by a step of claim 1, and its unencapsulated RNA is freely suspended within the MCC;
f) draining, by a MP, the MCC, inclusive of the viral pathogen's unencapsulated RNA, out of the patient's target lung and into an external vessel;
g) rinsing, by a MP, the MCC, inclusive of the viral pathogen's unencapsulated RNA, out of the patient's target lung and into an external vessel utilizing a continuous aqueous therapeutic solution;
h) determining, by a MP, utilizing a visualization and/or medical device that an initial volume metric of a MCC in an external vessel contains and acceptable or non-acceptable concentration of unencapsulated RNA of a viral pathogen;
i) repeating, by a MP, as necessary, a step of a claim 1 and/or 2 until a secondary or thereafter volume metric of a MCC in an external vessel contains an acceptable concentration of unencapsulated RNA of a viral pathogen; and
j) repositioning, by a MP, the patient on the medical table, and repeating a step of claim 1 and/or claim 2 on a second lung of the patient.

4) A process of claim 1 of performing, by a MP, a ternary phase of a ternary phased medical procedure upon the patient further comprising the steps of;
a) diagnosing, by a MP, as a result of conducting a post-procedural medical examination upon the patient, the patient's infectious status as negative for a viral pathogen;
b) rehabilitating, by a MP, the pulmonary system of the patient;
c) following-up, by a MP, post medical procedure with the patient, by checking and monitoring a vital sign of the patient, determining as required, a length of stay in a medical facility by a MP, prescribing, as required a medication by a MP, discharging as required, the patient from a medical facility and providing by a MP the patient with a post-procedural recovery instruction to follow, and determining by a MP, as required, a return visit by the patient to test an infectious status of the patient; and
d) recording, by a MP, by utilizing the computer and the network, a result of the FLiM in the CCNTCRMD for patient medical recording purposes and for clinical trial evaluation.
